(19) United States
(12) Patent Application Publication
(10) Pub. No.: US 2009/0204138 A1
(43) Pub. Date: Aug. 13, 2009

(54) LANCET HAVING A GROUND TIP AND METHOD FOR PRODUCING

(76) Inventor: Thomas Weiss, Mannheim (DE)

Correspondence Address: BOSE MCKINNEY & EVANS LLP, 111 Monument Circle, Suite 2700, INDIANAPOLIS, IN, 46204, US

(21) Appl. No.: 12/364,859

(22) Filed: Feb. 3, 2009

Related U.S. Application Data
(63) Continuation of application No. PCT/EP2007/006795, filed on Aug. 1, 2007.

(30) Foreign Application Priority Data
Aug. 5, 2006 (EP) EP 06 016 389.6

(51) Int. Cl. A61B 17/32; B24B 1/00

(52) U.S. Cl. 606/181; 451/54

(57) ABSTRACT

A lancet made of lancet wire having a ground tip. The lancet has a first ground face and a further ground face, which are located at a defined angle relative to one another, the length of the lancet being at most 16 mm. Also disclosed is a method for producing lancets for taking blood for medical-analytical purposes, which have at least one ground face that is generated in a grinding procedure. A lancet wire is positioned using a positioning device in a defined grinding position relative to a grinding element of a grinding device, and the following method steps are performed in this sequence: drawing off the lancet wire from a roll and transporting it into the positioning device; fixing or securing the lancet wire in the positioning device; grinding the free end of the lancet wire using the grinding device to generate at least one ground face; and cutting off the free end of the lancet wire in a cutting position to form a lancet having a defined length.

### RELATED APPLICATIONS

This application is a continuation of International Application PCT/EP2007/006795, filed on Aug. 1, 2007, which claims priority to EP 06016389.6, filed Aug. 5, 2006, which are hereby incorporated by reference in their entirety.

### BACKGROUND

The invention relates to a lancet for taking blood having a ground tip made of lancet wire, which has a first ground face and a further ground face. The two ground faces are located at a defined angle relative to one another. The invention also relates to a method for producing lancets which have a ground face.

Lancets for taking blood from a body part are used in medical analytics to generate a wound in a body part from which blood escapes. The lancets are preferably used in puncture devices or puncture aids, by means of which the patient can pierce himself.

Puncture aids of this type are used in the field of diabetes care. It is of particular medical significance here that the patients regularly check the blood sugar content of their blood. Only in this way is continuous and consistent monitoring of the insulin values of the patients possible, in order to allow an adapted, optimum setting of the insulin dose. Long-term consequences of strong blood sugar oscillations, such as blindness, can thus be avoided. Studies have shown that the patient's acceptance and frequency of use of puncture aids are a function of the piercing pain. A frequent check is only performed by the patients if the pain sensation upon piercing into the skin is as small as possible.

One factor which significantly affects pain sensation of the patient is piercing depth. To ensure an exactly reproducible puncture depth, the lancets are to have an exactly predetermined length. All new lancets must also have the same length upon changing of a lancet and upon replacement of a used lancet by a new one. The permissible length tolerance of the lancets is thus very small, which makes the production of lancets using prior art methods more costly.

The pain sensation of the patients is also significantly influenced by the quality of the ground tip. A three-facet grind of the lancet tip has proven to be especially suitable, in which a main face and two cutting faces inclined to one another are provided, so that an exact and fine tip is generated on the lancet end. A sharp cutting edge which cuts open the skin is formed in each case between the cutting faces and the lateral face of the lancet. During piercing, a small hole is generated in the skin by the tip in a first phase. Upon further penetration of the lancet into the skin, the cutting face cuts open the initially small opening further, so that blood can exit after the lancet is pulled out of the skin.

To produce lancets of this type, lancet wire is cut to the desired length and its tip is then ground. For this purpose, the lancet wire pieces are mounted on a clamping board on which they are held during the grinding procedure. To generate the individual ground faces, two clamping rails of the clamping board are shifted toward one another so that the individual lancets are rotated around their particular longitudinal axis. The rotational angle which can be achieved by means of such a configuration is in a range of less than 90°. Only at such small rotational angles is it possible to pivot a plurality of lancets located adjacent to one another uniformly around the same angular position. Both the cutting off of the lancet wire pieces and also the clamping in the clamping board must be performed with high precision. Only in this manner can lancets be produced having exact length and low manufacturing tolerance. The very precise procedure in the two production steps makes the overall production of the lancets time-consuming and costly. Mass production is only possible with difficulty. The proportion of manual interventions and monitoring in the production process is relatively high.

Because diabetes patients must puncture themselves several times a day to be able to perform a continuous check of their blood sugar values, puncture devices which contain a lancet magazine having a plurality of lancets are being used more and more frequently. In order that the patient may carry along the puncture device with them comfortably, it must be as small as possible. This results in the requirement that the lancets used must also be as small and short as possible. The length of the lancets which can be produced using the method described above known until now is limited to a minimum dimension, however. In practice, producing lancets having a length less than 16 mm is not possible, above all because the lancets can no longer be held exactly during the grinding procedure in the clamping board used as the rotational and positioning device.

For hygienic reasons, lancets are typically used only once. Therefore, effort is made to produce them as cost-effectively as possible. This results in the desire for automation of the production process to be able to produce in large batches.

### SUMMARY OF THE INVENTION

The present invention provides an improved lancet having a ground tip which is suitable for use in compact and small puncture devices and which can also be produced in large batches.

The lancet according to an exemplary embodiment is made of lancet wire and has a ground tip and comprises a first ground face and at least one further ground face, the faces being located at a defined angle to one another. The lancet has a two-facet grind or, in the event of a plurality of further ground faces, a multifaceted grind. The length of the lancet according to the invention is at most 16 mm, whereby it is outstandingly suitable for use in very compact puncture devices.

The lancet according to exemplary embodiments may have a length of at most 12 mm. In an especially preferred embodiment, the length of the lancet is smaller than 10 mm. For using the lancet in puncture devices having a very small overall size or in magazines having a large number of lancets, the maximum length of the lancet can also be at most 5 mm.

In addition to a lancet having a two-facet grind, a lancet having a three-facet grind is also preferred, whose length can also be less than or equal to 16 mm, for example, the lancet can be shorter than 12 mm or 10 mm, preferably at most 5 mm.

These teachings also provide a method for producing lancets for taking or withdrawing blood for medical-analytical purposes, which have a first ground face, which is generated in a least one grinding procedure. A lancet wire is positioned by means of a positioning device in a defined grinding position in relation to a grinding element of a grinding device. To produce the lancets, the lancet wire is drawn off of a roll and is transported into the positioning device in which it is subsequently fixed. In a further step, the free end of the lancet wire is ground using a grinding device to generate the ground face. The free end of the lancet wire is first cut off at a cutting position after the grinding of the ground face, whereby a lancet with a defined length results.

Thus, according to this method, in contrast to the known methods, a free end of the lancet wire is first ground and the lancet wire is subsequently cut off in the desired length. In this manner, lancets with a very precise length can be produced. The length tolerances are very small. For example, if the lancet wire is fed too far forward, so that the distance between the free end of the lancet wire and the cutting position is greater than the desired length of the lancet, the free end of the lancet wire is ground off so far by the grinding device that the length results, which is set between the grinding device and the cutting position. The length of the lancet produced is thus not a function of the feed of the lancet wire, but rather only of the cutting position and its distance from the grinding device. The desired length of the lancets can be set by simple adjustment.

Because the lancet wire is unwound from a roll or supply, it is provided as a quasi-endless wire. As soon as the free end of the lancet wire is ground and the lancet is cut off after the grinding, the lancet wire is transported further in the direction of the positioning device. The grinding of the new free end of the lancet wire can thus begin immediately after a single simple preparation step. Such a procedure can be easily automated. The lancet wire can be supplied at high speed. Exact positioning is not necessary because it has no influence on the later length of the lancet.

The method steps of drawing off the lancet wire, transporting and fixing (securing), grinding, and subsequent cutting off are performed in the specified sequence. Of course, this does not preclude that further additional steps can be performed before, after, or between the cited method steps.

The method is typically used for producing a lancet with a ground tip. The lancet has a first ground face and at least one further ground face, which are oriented at a defined angle relative to one another. The free end of the lancet wire is ground using the grinding device to generate the plurality of ground faces. One grinding face at a time is generated in a plurality of grinding procedures and the lancet wire is rotated between the grinding procedures into a grinding position corresponding to the next grinding face to be generated, the rotational movement having an axial rotational movement component relative to the longitudinal axis of the lancet wire. In the simplest case, the rotational movement only has an axial component, i.e., the lancet wire is only rotated around its longitudinal axis. Of course, all other movements are also comprised by this, as long as a rotation around the longitudinal axis also occurs. In this way, the lancets having a ground tip and a plurality of ground faces can be produced. A lancet having a three-facet grind, which has a main ground face and two further ground faces referred to as cutting faces, is especially preferred.

To rotate the lancet wire into the corresponding grinding positions, the positioning device is rotated. Alternatively, the grinding device can also be rotated in relation to the positioning device. However, a positioning device which has at least one rotational element to rotate the lancet wire can be employed.

In one embodiment of the invention, the positioning device is adapted to hold a plurality of lancet wires. The lancet wires are located adjacent to one another in a plane which runs transversely to the longitudinal direction of their free ends. The lancet wires are also preferably fixed in this plane during the grinding procedures.

The lancet wires are positioned adjacent to one another in such a manner that a plurality of lancet wires can be ground simultaneously. A plurality of ground faces can thus be generated in parallel in one work step. The piece count during the production of the lancets is thus increased. Of course, the lancet wires may also be ground in sequence.

In a further exemplary method, the grinding element is moved during the grinding procedure in such a manner that the free ends of a plurality of lancet wires fixed in the positioning device are ground in sequence during the movement. The grinding element is preferably moved along the lancet wires positioned adjacent to one another so that the lancet wires are ground in sequence, wherein a plurality of wires can also be ground simultaneously, meaning that the grinding of one lancet wire can begin before the grinding procedure has ended on one or more preceding lancet wires.

In large-batch manufacturing, the positioning device can have a width of 2 to 3 m, so that several hundred lancet wires can be positioned adjacent to one another. In this manner, a very cost-effective mass production of lancets having a ground tip can be implemented.

After all lancet wires retained and/or clamped in the positioning device have been processed in a grinding procedure, a first ground face having been produced, the lancet wires fixed in the positioning device are moved, typically simultaneously, into a further grinding position, which corresponds to a further ground face. The grinding element is then again moved past the lancet wires in such a manner that a further grinding procedure is performed on the free ends of the lancet wires during the movement of the grinding element.

An advantage of the exemplary method is that the relatively short lancet does not already have to be fixed and retained during grinding, but rather a longer piece of the lancet wire is retained on its free end, the free end not having to be cut off, i.e., still being connected to the roll. A very solid, robust, and reliable fixing of the wire is thus possible. In this manner, the exact and precise positioning of the free end of the lancet wire (in the direction transverse to its longitudinal axis) is also improved, which results in very high manufacturing quality and product quality and allows small manufacturing tolerances.

In an exemplary embodiment of the method, the free end of the ground lancet wire is cut off in such a manner that the length of the resulting lancet from its tip to the cutting point is at most 20 mm, preferably at most 16 mm. The free end of the lancet wire is typically cut off in such a manner that the resulting lancet has a length of at most 5 mm. Therefore, significantly shorter lancets can be produced using the method than is possible using known automated methods. The method according to these teachings therefore forms the basis for an automated mass production in large batches of short lancets having a ground tip and a length of at most 16 mm, preferably at most 12 mm, especially preferably at most 10 mm.

The method is suitable for producing lancets having a diameter from approximately 0.2 mm to 0.8 mm. The lancet can comprise solid material. However, it is also possible to implement lancets which are at least partially hollow. Lancets produced by the method can contain a capillary channel, which can also be arranged as a capillary groove open on one side.

Further details and features of the invention will become clear from the following description of illustrative embodiments. The respective features can be embodied either singly or in several combinations with one another, though the invention is not limited to the illustrative embodiments.

### BRIEF DESCRIPTION OF DRAWINGS

The above-mentioned aspects of the present invention and the manner of obtaining them will become more apparent and the invention itself will be better understood by reference to the following description of the embodiments of the invention, taken in conjunction with the accompanying drawings, wherein:

FIG. 1 is a schematic diagram in perspective view of a device for producing lancets having a ground tip;

FIG. 2 is a fragmentary perspective view of the device from FIG. 1;

FIG. 3 is a sectional view through a part of a rotating and positioning device of the device from FIG. 1;

FIGS. 4-6 are sectional views of the rotating and positioning device from FIG. 3 during various method steps;

FIG. 7 is a view of a lancet produced using the method according to the invention;

FIG. 8 is a cross-section along line A-A of the lancet of FIG. 7;

FIG. 9 is a cross-section along line B-B of the lancet of FIG. 7;

FIG. 10 is a view of an alternative embodiment of a lancet produced using the method according to the invention; and

FIG. 11 is a cross-section through the lancet of FIG. 10.

The illustrative embodiments are shown schematically in the figures. Identical reference numbers in the individual figures designate elements which are identical or whose functions are identical, or which correspond to one another in terms of their function.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

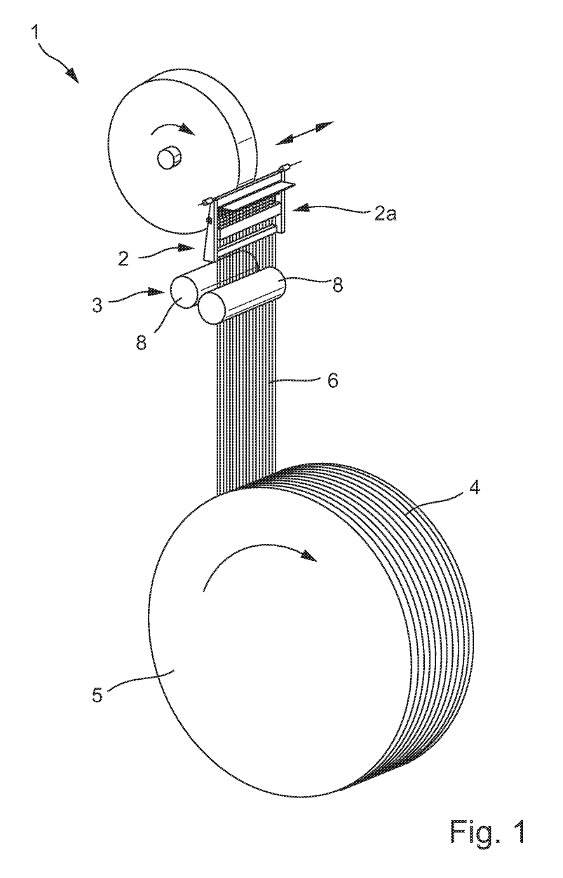

The device for producing a lancet having a ground tip according to FIGS. 1, 2, and 3 comprises a grinding device 1, a positioning device 2a, and a transport device 3. The positioning device 2a is arranged as a rotating and positioning device 2, by means of which the lancet wires 6 can be rotated and positioned. The transport device 3 is positioned between the rotating and positioning device 2 and a roll 5, implemented as a winding drum 4, on which a plurality of lancet wires 6 located adjacent to one another are wound.

Using the transport device 3, the lancet wires 6 located in parallel are drawn off of the winding drum 4, transported in the direction of the rotating and positioning device 2, and supplied thereto. Each time the free end of the lancet wire 6 has been processed and cut off, the lancet wire 6 is fed further into the positioning device 2. The transport device 3 has two oppositely rotating transport rollers 8 for this purpose, whose rotation causes a transport of the lancet wires 6. Alternatively, the transport device 3 can comprise a clamping device, which is moved translationally by a predetermined stroke to supply the lancet wire 6 to the positioning device 2a. The transport device 3 can be pneumatically driven to implement the feed.

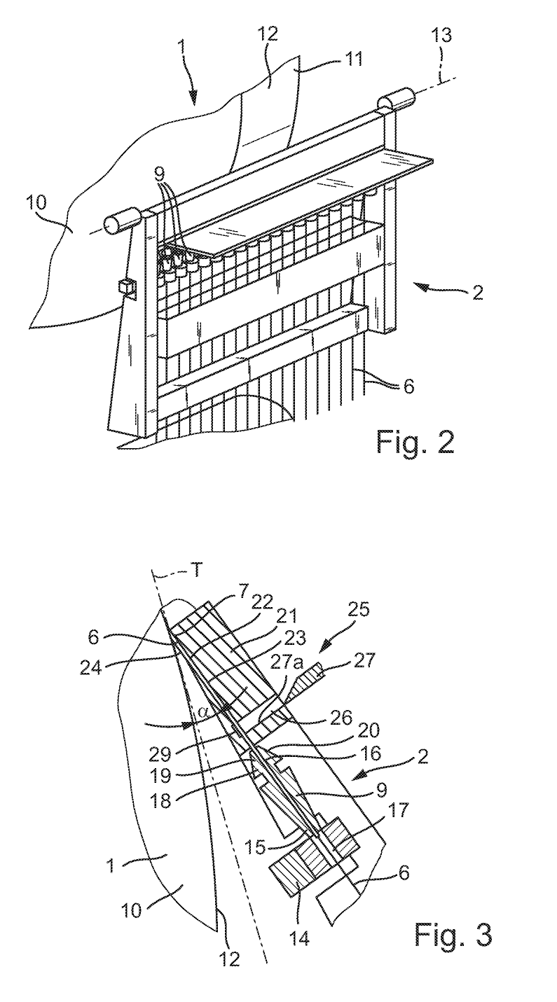

The rotating and positioning device 2 shown in detail in FIG. 2 has a frame-type support construction. It comprises a plurality of rotational elements 9, which are located adjacent to one another in a row. Each rotational element 9 holds one lancet wire 6 at a time and fixes it in the rotating and positioning device 2.

The grinding device 1 has a grinding element 10, which is arranged here as a cylindrical grinding disc 11. The grinding element 10 can also be a grinding roller. The lateral face of the grinding disc 11 is used as the grinding face 12, which grinds the free ends 7 of the lancet wires 6 during the rotation of the grinding disc 11 around its central axis. During the rotation of the grinding disc 11, the grinding device 1 is moved transversely to the longitudinal axis of the free ends 7 of the lancet wires, so that the rotating and positioning device 2 is traveled along its width. All lancet wires 6 clamped in the rotating and positioning device 2 are thus ground in sequence (a plurality of lancet wires 6 can be processed simultaneously). The movement of the grinding element 10 preferably occurs along the width of the rotating and positioning device 2 in such a manner that the grinding element 10 is moved beyond the ends of the rotating and positioning device 2.

The position of the lancet wires 6 in relation to the grinding device 1 and/or its grinding face 12 is changed by inclining the rotating and positioning device 2. For this purpose, the rotating and positioning device 2 is pivoted around a rotational axis 13 running in the area of the free ends 7. In this way, ground faces with different angles of inclination can be generated on the lancet wires 6. The angle of inclination of the free end of the lancet wires 6 can be changed in relation to the grinding face 12 of the grinding element 10 between at least two of the grinding procedures, so that at the end of the production process, the free end of the lancet wire 6 has at least two ground faces having different angles of inclination.

In addition to the inclination of the lancet wires 6 in relation to the grinding face 12, their rotational angle in relation to the longitudinal axis of the wires can also be changed. For this purpose, each individual lancet wire 6 is rotated by the rotation element 9 of the rotating and positioning device 2 in which the lancet wire 6 is fixed by rotating the rotational element 2 around the longitudinal axis of the lancet wire 6.

The rotation element 9 is positioned by means of a retainer 14 on the rotating and positioning device 2. It is preferably implemented as a rotationally-symmetric round element whose lower part 15 has a conical shape. A through hole 16 is used to accommodate and guide the lancet wire 6. For simpler insertion of the lancet wire 6 into the rotation element 9, a lower opening 17 of the through hole 16 is expanded so that the opening diameter is a plurality of times greater than the diameter of the lancet wire 6.

A least a part of the rotation element 9 is especially preferably segmented into at least three segments 18 in the longitudinal direction of the lancet wire 6. The lancet wire 6 is fixed and retained by compressing the segments 18 in the direction of the axis of the lancet wire 6. In the embodiment shown in FIG. 3, the segments 18 are located in the front (on top in the figure) part 19 of the rotation element 9. The segments 18 are adapted as fixing jaws 20, which clamp the wire, so that it can be rotated around its own axis, i.e., its longitudinal axis, in its fixed position by rotating the rotation element 9.

As the lancet wire 6 is fed through the transport device 3, the fixing jaws 20 are loosened so that the wire can be moved. For this purpose, the rotation element 9 can be arranged similarly to the lead holder of a mechanical pencil, wherein the fixing jaws 20 can be opened and/or closed by a movement in the longitudinal direction of the lancet wire 6.

The rotating and positioning device 2 comprises a support element 21, which is located in the transport direction of the wire in front of the rotation element 9 and which offers the free end of the lancet wire 6 a support face 22, against which the lancet wire 6 presses during the grinding procedure. The support element 21 has a guide channel 23, which runs at an inclined angle through the support element 21 in such a manner that the guide channel 23 has an open end 24, on which the support face 22 is located.

A cutting unit 25, which comprises a groove 26 and a cutting blade 27, which is guided in the groove 26, is integrated in the support element 21. The groove 26 is located in the support element 21 in such a manner that the guide channel 23 intersects the groove 26. A bottom face 29 of the groove 26 is flush with the guide channel 23. During the cutting off of the lancet wire 6, the bottom face 29 forms a contact and positioning face for the lancet wire 6, which extends through the guide channel 23. The distance of the groove 26 from the upper end of the support element 21 is selected in such a manner that it corresponds to the desired lancet length. In the example shown, the distance is 12 mm. By cutting off the free end of the lancet wire 6 using the cutting blade 27 at a cutting point 27a, a lancet 28 with the desired predefined length of 12 mm is generated. Instead of using the cutting blade 27 shown here, the free end of the lancet wire 6 can be cut off using a chopping blade, a cutting disc, or a laser. Of course, further known possibilities for cutting through a wire are also suitable.

It can be seen from FIG. 3 that the lancet wire 6 can be cut off at a point which lies between the rotation element 9 and the ground tip of the lancet wire 6. In other words, the cutting point 27a lies between the rotation element 9 and the open end 24 of the guide channel 23. The free end 7 of the lancet wire 6 is preferably cut off while the lancet wire 6 is fixed in the rotation element 9. In this manner, the lancet wire 6 is held exactly in its position, so that a smooth main face arises and the non-ground end of the lancet 28 does not have to be post-processed. The production costs during the production of the lancets 28 are thus decreased.

The lancet wire 6 is cut off between the rotation element 9 and the ground tip 30. However, it can also be advantageous to cut through the lancet wire behind the rotation element 9 viewed from its tip. From manufacturing aspects this can be a suitable variant in certain embodiments.

The angle of inclination α between the grinding face 12 and the free end 7 of the lancet wire 6 can be changed by pivoting the rotating and positioning device 2. This “grinding angle” corresponds to the angle between the axis of the free end of the lancet wire 6 and the tangent T of the grinding face 12 formed in the grinding point. Because of the size of the grinding element 10 relative to the diameter of the lancet wire 6 and/or its tip, the curvature of the grinding element 10 can be neglected, so that in a first approximation the tangent of the grinding face 12 in the grinding point can be set equal to the grinding face 12 itself.

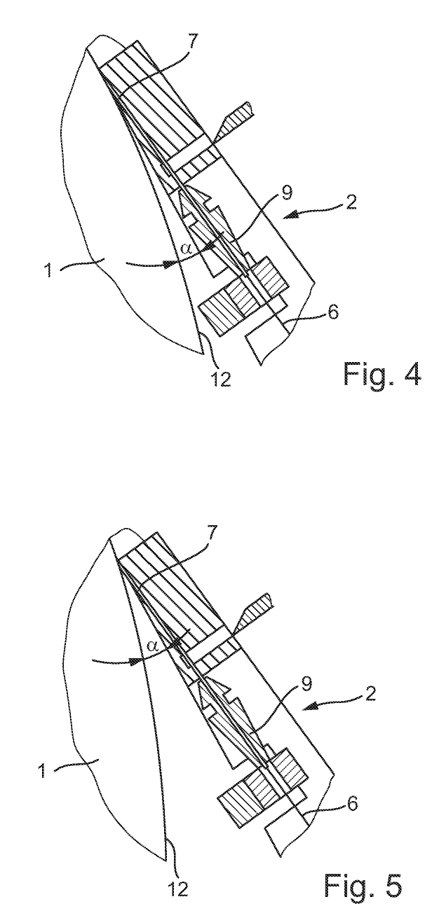

The production process is described with reference to FIGS. 4, 5, and 6. FIG. 4 shows the position of the rotating and positioning device 2 in relation to the grinding device 1 during the first grinding procedure. The lancet wire 6 has been supplied by the transport device 3 of the rotating and positioning device 2 and fixed or secured in the rotation element 9. The angle of inclination between the grinding face 12 and the free end 7 of the lancet wire 6 is 7.5° here. The grinding disc 11 grinds the free end 7 of the lancet wire 6 at the angle of inclination, so that a first ground face 31 is generated, which is referred to as the main ground face and preferably runs at an angle of 7.5° to the longitudinal axis of the lancet wire 6.

During the grinding procedure, the free end 7 of the lancet wire 6 is supported by the support element 21, which is a component of the rotating and positioning device 2. The free end of the lancet wire 6 to be ground presses against the support face 22, so that it can not be bent by the grinding element 10 and smooth, flat ground faces arise.

In at least one of the grinding procedures, the angle of inclination a between the grinding face 12 and the axis of the lancet wire 6 preferably has a value between 5° and 10°. The angle of inclination a is especially preferably between 7° and 8°.

After all lancet wires 6 have been ground in the rotating and positioning device 2 in a first grinding procedure, so that they have the main ground face 32, the rotating and positioning device 2 is pivoted and the angle of inclination α is changed. The new angle of inclination α between the grinding face 12 and the axis of the lancet wire 6 preferably lies between 15° and 25°, especially preferably between 17° and 20°. This angle of inclination α is set up in at least one of the other grinding procedures, and preferably remains unchanged during the third grinding procedure. In the example shown in FIG. 5, the angle of inclination α is equal to 18°.

Before the grinding device 1 begins with the next grinding procedure to generate a further ground face 33, the lancet wire 6 is rotated by the rotation element 9 around its longitudinal axis. A rotation having an angle of rotation β between approximately 15° and approximately 25° is preferred, and an angle of rotation of β=20° is especially preferred.

At the end of the second grinding procedure, the free ends of the lancet wires 6 have a further ground face 33, which is referred to as the first cutting face, in addition to the main ground face 32.

After the second grinding procedure, all lancet wires 6 of the rotating and positioning device 2 are rotated by the particular rotation elements 9 around their own axis. This second rotation is performed opposite to the rotational direction of the first rotation. The angle of rotation β′ is preferably between approximately 30° and approximately 50°, especially preferably 40°. The angle of rotation β′ corresponds to double the angle of rotation β of the first rotation.

After the rotation of all lancet wires 6, the grinding device 1 is again moved past the rotating and positioning device 2, so that a further ground face 33 is generated on the free ends 7 of the lancet wires 6 in a third grinding procedure. As a result of this third grinding procedure, the free end 7 of the lancet wire 6 has a second cutting face 35 in addition to the main ground face 32 and the first cutting face 34.

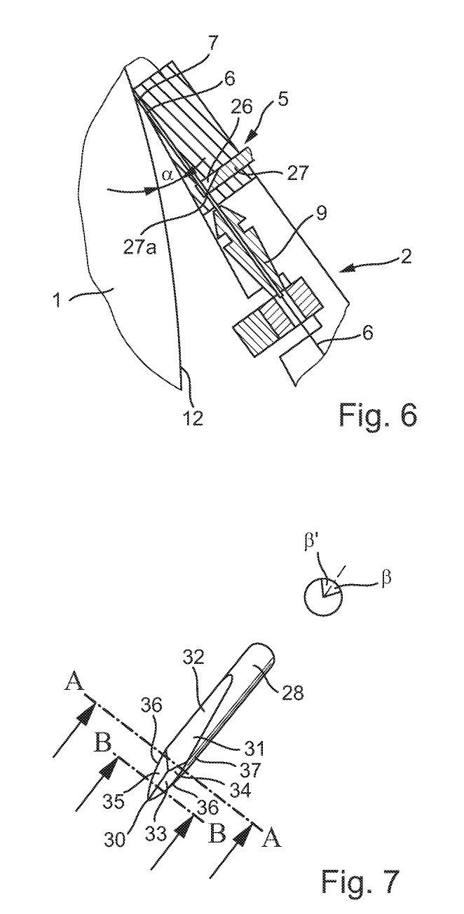

After all free ends of the lancet wires 6 clamped in the rotating and positioning device 2 have been ground and all ends have a three-facet grind, the cutting blade 27 of the cutting unit 25 is moved in the groove 26 toward the lancet wire 6 (FIG. 6). The cutting blade 27 is located at the cutting position 27a and cuts off the free end 7 of the lancet wire 6 at a point between its tip 30 and the rotation element 9. The lancet 28 shown in FIG. 7 with a length of 12 mm and with a ground sharp tip 30, which generates a puncture in the skin of a body part with little pain, thus arises.

The lancet 28 has the desired three-facet grind having a main ground face 32 and two cutting faces 34, 35. The two cutting faces 34, 35 form a cutting edge 36 with the lateral face 37, using which the wound is cut open upon piercing of the lancet 28 into the skin of a body part, so that blood can exit from the wound after the lancet is withdrawn.

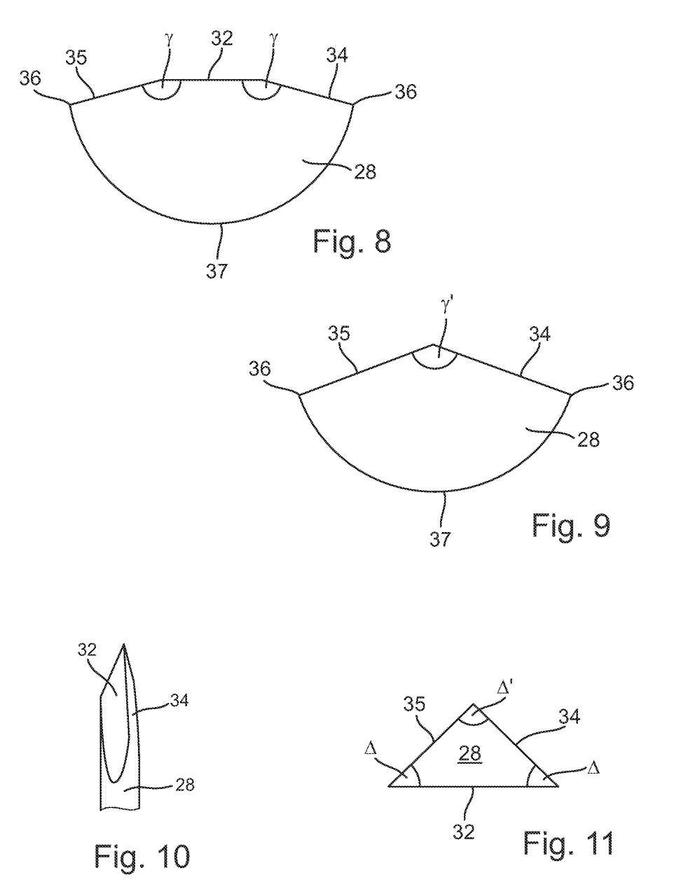

The intersection angles shown in FIGS. 8, 9, and 11 represent the angle between the intersection lines of the corresponding faces lying in a sectional plane perpendicular to the longitudinal axis of the lancet 28. The angles γ, γ′, Δ, and Δ′ shown are thus formed between the section lines of the corresponding faces resulting in a section perpendicular to the longitudinal axis of the lancet wire.

FIG. 8 shows a cross-section through the lancet along line A-A. The lancet 28 has an angle γ between the main ground face 32 and each of the cutting faces 34 and 35. The angle γ is 180°−β, wherein β is the angle of rotation. In the example shown, the angle γ is equal to 160°.

The two cutting edges 36, which are formed in each case between the cutting faces 34 and 35 and the lateral face 37, can be seen clearly.

A further section through the lancet 28 is shown in FIG. 9. This section is located closer to the tip of the lancet 28. The two cutting faces 34 and 35 directly abut one another here and form an angle γ′. The angle γ′ can be calculated as γ′=180°−2β or γ′=180°−β′, wherein β is the angle of rotation of the first rotation and wherein β′ is the angle of rotation of the second rotation of the rotation element 9 around the axis of the lancet wire 6. In the example shown here, the angle γ′ is equal to 140°.

In addition to the lancet shape described in FIGS. 7 through 9, it is also possible using the method according to the invention to produce lancets 28 in which the main ground face 32 is opposite to the two cutting faces 34, 35. A lancet of this type is shown in FIG. 10.

To illustrate the configuration of the main ground face 32 and the cutting faces 33, 34, a section through the lancet 28 according to FIG. 10 is shown in FIG. 11. An angle Δ is formed in each case between the main ground face 32 and the cutting faces 34, 35, the angle A is approximately 20° in the example shown. To produce a lancet of the shape shown here, after the grinding of the main ground face 32, the lancet wire 6 is rotated by approximately 160° or 200° respectively before the cutting face 34 or 35 is ground. After the grinding of the cutting face 34 or 35, the lancet wire 28 is rotated once again by 40°. An angle Δ′ of 140° thus arises between the cutting faces 34, 35. The angle A′ results from 1800 minus the angle of rotation by which the lancet wire is rotated between the two grinding procedures. Of course, other A are also possible, which preferably lie in the range from 10° to 30°, especially preferably in the range from 15° to 25°.

This grinding shape, which is referred to as a “relief-ground lancet,” is therefore possible because the lancet wire can be rotated by approximately 270° around its own axis, without having to worry about the lancet wire slipping through in the rotation element 9.

Lancet wire which comprises metal, preferably steel, is used to produce the lancets. Alloys of metals are also suitable. The diameter of the lancet wire is approximately 0.2 to 0.8 mm, preferably 0.3 mm.

While exemplary embodiments incorporating the principles of the present invention have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A lancet for sampling blood for medical-analytical purposes, comprising:
a ground tip formed from lancet wire;
the ground tip comprising a first ground face and a further ground face located at a defined angle relative to one another; and
wherein, the length of the lancet is at most 16 mm.

2. The lancet of claim 1, wherein the length of the lancet is at most 12 mm.

3. The lancet of claim 1, wherein the length of the lancet is at most 10 mm.

4. The lancet of claim 1, wherein the length of the lancet is at most 5 mm.

5. A lancet for sampling blood for medical-analytical purposes, comprising:
a ground tip formed from lancet wire;
the ground tip comprising a main ground face and two cutting faces, the main ground face forming an angle γ with each cutting face of between 155° and 165°; and
wherein, the length of the lancet is at most 16 mm.

6. The lancet of claim 5, wherein the cutting faces enclose an angle γ′ which is between 130° and 150°.

7. The lancet of claim 5, wherein the main ground face is located opposite to the two cutting faces, wherein the main ground face forms an angle Δ with each cutting face of less than 90°.

8. The lancet of claim 5, wherein the main ground face is located opposite to the two cutting faces, wherein the main ground face forms an angle Δ with each cutting face of less than 20°.

9. The lancet of claim 5, wherein the cutting faces enclose an angle Δ′ which is between 120° and 160°.

10. A method for producing lancets of the type having a ground face, comprising:
(a) drawing a lancet wire from a supply and transporting the lancet wire into a positioning device;
(b) positioning the lancet wire in the positioning device in a defined grinding position relative to a grinding element of a grinding device;
(c) securing the lancet wire in the positioning device;
(d) grinding the free end of the lancet wire with the grinding device and thereby generating the ground face; and
(e) cutting off the free end of the lancet wire in a cutting position to form a lancet having a defined length.

11. The method of claim 10, further comprising, after the step of grinding the free end of the lancet:
repositioning the lancet wire in the positioning device; and
again grinding the free end of the lancet, thereby generating a second ground face, the ground face and the second ground face being oriented at a defined angle relative to one another.

12. The method of claim 11, further comprising rotating the lancet wire between the step of grinding the free end of the lancet and again grinding the free end of the lancet.

13. The method of claim 12, wherein the rotating has an axial rotational component.

14. The method of claim 11, further comprising changing the angle of inclination between the free end of the lancet wire in relation to a grinding face of a grinding element between the step of grinding the free end of the lancet and the step of again grinding the free end of the lancet.

15. The method of claim 11, further comprising securing the lancet wire in a rotation element of the positioning device and rotating the lancet wire around its longitudinal axis between the grinding step and the again grinding step.

16. The method of claim 15, wherein at least a part of the rotation element is segmented into at least three segments in the longitudinal direction of the lancet wire and the lancet wire is fixed in the direction of the longitudinal axis of the lancet wire by compressing the segments.

17. The method of claim 15, further comprising cutting off the lancet wire at a position between the rotation element and the ground tip of the lancet wire.

18. The method of claim 15, further comprising cutting off the free end of the lancet wire while the lancet wire is fixed in the rotation element.

19. The method of claim 11, wherein, in at least one of the grinding procedures, the angle of inclination between a grinding face of a grinding element and the longitudinal axis of the lancet wire is between 5° and 10°.

20. The method of claim 19, wherein, in the other grinding procedure, the angle of inclination between the grinding face and the longitudinal axis of the lancet wire is between 15° and 25°.

21. The method of claim 10, further comprising supporting the free end of the lancet wire with a support element during the grinding.

22. The method of claim 10, further comprising positioning and securing a plurality of lancet wires adjacent to one another in the positioning device in a plane running transversely to the longitudinal direction of free ends of the lancet wires.

23. The method of claim 22, further comprising moving the grinding element during a grinding procedure such that the free ends of the plurality of lancet wires secured in the positioning device are ground in sequence during the movement.

24. The method of claim 22, wherein the free ends of the plurality of lancet wires are ground simultaneously by the grinding element.

25. The method of claim 10, wherein the free end of the lancet wire is cut off such that the length of the resulting lancet from its tip to the cutting point is at most 16 mm.

26. The method of claim 10, wherein the free end of the lancet wire is cut off using a chopping blade, a cutting blade, a cutting disk, or a laser.

27. The method of claim 10, further comprising, between the step of grinding the free end of the lancet and again grinding the free end of the lancet, rotating the free end of the lancet wire by a defined angle of rotation in a range from 90° to 270°.

28. The method of claim 27, further comprising, between the step of grinding the free end of the lancet and again grinding the free end of the lancet, rotating the free end of the lancet wire by a defined angle of rotation in a range from 150° to 210°.

28. The method of claim 27, further comprising, between the step of grinding the free end of the lancet and again grinding the free end of the lancet, rotating the free end of the lancet wire by a defined angle of rotation in a range from 160° to 170°.

29. The method of claim 27, further comprising, between the step of grinding the free end of the lancet and again grinding the free end of the lancet, rotating the free end of the lancet wire by a defined angle of rotation in a range from 190° to 200°.
